(19) United States
(12) Patent Application Publication
(10) Pub. No.: US 2022/0184856 A1
(43) Pub. Date: Jun. 16, 2022

(54) METHOD FOR RECYCLING GLASS FIBRE REINFORCED PLASTIC

(71) Applicant: LANXESS Deutschland GmbH, Cologne (DE)

(72) Inventors: Michael Witt, Eckersdorf (DE); Detlev Joachimi, Krefeld (DE)

(73) Assignee: LANXESS Deutschland GmbH, Cologne (DE)

(21) Appl. No.: 17/604,114

(22) PCT Filed: Apr. 6, 2020
(86) PCT No.: PCT/EP2020/059789

§ 371 Date: Oct. 15, 2021

(30) Foreign Application Priority Data
Apr. 15, 2019 (EP) 19169215.1

(51) Int. Cl. B29B 17/04

(52) U.S. Cl. CPC B29B 17/04; B29K 2077/00; B29B 2017/0496; B29B 2017/042

(57) ABSTRACT

The present invention relates to a process for recycling glass fiber-reinforced plastics, in particular plastics based on polyamide, polybutylene terephthalate or polyethylene terephthalate, to recover both the monomers of the polymer and the glass used for the glass fibers.

The present invention relates to a process for recycling glass fiber-reinforced plastics, in particular plastics based on polyamide, polybutylene terephthalate or polyethylene terephthalate, to recover both the monomers of the polymer and the glass used for the glass fibers.

### PRIOR ART

Modern, high resilience composite plastics are nowadays often based on glass fiber-reinforced thermoplastics, in particular polyamides or polyesters based on terephthalic acid. LANXESS Deutschland GmbH, Cologne, markets plastic pellet materials with chopped glass fiber reinforcement under the trade names Durethan® and Pocan® and markets continuous fiber-reinforced semifinished products/composites under the trade name TEPEX®. The mass fraction of glass fiber reinforcement in the marketed pellet materials is typically in the range from 5 to 80 percent by weight.

Glass-based fillers and reinforcers, in particular in the form of fibers, achieve considerable improvements in strengths, toughnesses and stiffnesses compared to a plastic component without filler or reinforcer. In recent years this has allowed substitution of many metal-based constructions by glass fiber-reinforced plastics (GRP), in particular in automaking.

Additional heat-stabilizing additives now even allow the use of GRP in regions subjected to high thermal stress which would be impossible for the pure polymer not stabilized with these additives, in particular in the field of motor vehicle engine bays.

GRP-based components nowadays allow cost-effective lightweight construction in the entire transport sector. The achieved weight reduction in motor vehicles allows energy consumption (fuel or electrical energy) to be significantly reduced.

However, at the end of a usage period, which in the case of a motor vehicle ends with it being sent to an auto recycler, GRP-based components place very high demands on a recycling operation, in particular when the intention is to replace new material composed of the same substance. This applies all the more to GRP components that must be highly additized for the target application in order to avoid or reduce downgrading during use. In the context of the present invention downgrading is to be understood as meaning a deterioration in the level of mechanical properties, in particular as a result of cleavage of the molecular chains of the (matrix) polymer.

Impurities also play an important role in the quality level of GRP-based plastic recyclates. Mechanical recycling places particularly high demands on the plastic waste to be recycled when the recyclate is to be used for production of the same or other demanding applications. Mechanical recycling employs only physical processes. Physical processes include for example washing, drying, comminuting, melting, compounding, melt filtration and re-pelletization. There are of course examples where plastic waste may be recycled into good-quality recyclate via physical processes. The following prerequisites are usually necessary:

The components made of GRP must be collected in type-identical fashion, the degree of soiling should be low, during the usage period no marked polymer degradation should have taken place and in addition over the course of the entire usage period only very few foreign substances should have been absorbed by the polymer matrix, for example special oils and their additizations, for example in the case of engine and transmission oil pans, for example for cars or trucks, or coolants in the case of cooling circuit components such as coolant reservoirs. Such plastic-based recyclate materials may be reused for producing new components using customary injection molding machines.

However, it has been found that mechanical recycling using purely physical processes results in a quality level equal to that of the original virgin product only in the rarest of cases.

The following are aggravating aspects for GRP which favor downgrading and thus hamper mechanical recycling, in particular via purely physical processes:
a high content of chopped glass fibers prevents the use during compounding of melt filters which especially in the case of continuous cleaning and discharging of separated impurities play a decisive role in the quality improvement of the obtained recyclates in mechanical recycling of unfilled thermoplastics.
during compounding, preferably with the customary corotating twin-screw extruders typically used therein, glass fibers are mechanically shortened, an effect which has a direct adverse impact on the strength and toughness of the GRP/compounds/composite.
Additives, in particular flame retardants or heat stabilizers, undergo alteration during the usage period of a GRP, in particular at high sustained use temperatures. However, the degradation products of such additives remain in the mechanical recycling circuit and thus in the GRP recyclate.

There are of course a multiplicity of proposals to counter downgrading in the mechanical recycling of GRP using physical methods. For example, the effect of a reduced fiber length in the recyclate may be compensated by subsequent addition of longer fibers. However, this process is naturally not suitable for continuous-fiber reinforced composite materials and is therefore limited only to chopped glass fiber-reinforced GRP.

However, polymer degradation may also be countered in numerous ways via targeted additization, in particular via chemically activated chain extension.

However it must be noted that downgrading is a fundamental problem in mechanical recycling of GRP and repeated passage through the usage-recycling circuits is not possible for GRP without marked effects on the quality and the product properties, in particular in terms of toughness, strength, stiffness, creep, heat resistance etc.

As mentioned, re-compounding reduces the fiber length. It is generally also not possible to directly remove the chopped glass fibers from the (matrix) polymer, which is highly viscous in the molten state, and thus separate the fibers from the polymer matrix.

The high cost and complexity of separating the fibers from the matrix, optionally subjecting them to further cleaning and preparing them for use as recyclate fibers must ultimately also be compared to the low cost of virgin glass fiber.

All three reasons explain why glass fibers from postconsumer GRP materials have hitherto only seldom been recycled and sent for re-use as a filler or reinforcer.

WO 2017 007965 A1 describes a process for depolymerization of unreinforced polyethylene terephthalate to obtain terephthalic acid and ethylene glycol therefrom. To this end the polymer is added to a mixture of a nonpolar solvent which swells the polymer and a reagent which cleaves the ester functionality and is depolymerized. WO 2017 007965 A1 does not elaborate on the recycling of fillers or reinforcers, in particular of glass fibers.

EP 3 023 478 A2 discloses a process which makes it possible to recover the fibers, especially in the case of carbon fiber composite plastics. This comprises initially pyrolyzing the polymer matrix of the composite plastic in a main reactor at 400-600° C. The remaining fiber residue with soot residues is washed, thus causing the fibers to adsorb water. The moist residue is subsequently returned to the main reactor, wherein the fibers are cleaned under oxidizing conditions at 350-400° C. The cleavage products formed in the first step during the pyrolysis are not subjected to material recovery but rather are transferred to a second reactor where a thermal plasma is used to neutralize the toxicological cleavage products at up to 15 000° C.

JP 2000034363A describes a process for depolymerization of chopped glass fiber-reinforced polyamide 6 composite plastics wherein the polymer matrix is initially depolymerized at temperatures around 280° C. and then the entire reaction mixture is added to water and the caprolactam obtained by depolymerization—i.e. the monomer building block of polyamide 6 -is is dissolved in water. Due to the markedly lower viscosity of the aqueous caprolactam solution of often only 1-100 mPa·s the glass fibers may be removed from the continuous water phase and washed.

The disadvantage of the process of JP 2000034363A is the energy-intensive distillation to obtain high-purity caprolactams from the aqueous, dilute caprolactam solutions. In addition, the reuse of the glass fibers separated and washed in JP 2000034363A is not without problems. Due to the shortening of the fiber lengths effected in the processing the removed and washed glass fibers from JP 2000034363A no longer achieve the same reinforcing effect as the use of virgin glass fiber. In addition, composite plastics nowadays contain a large number of additives that remain on the fibers and cannot be completely washed off with water. In these cases the process according to JP 2000034363A then does not supply high-quality recyclate fibers either.

Finally, for optimal functioning glass-based reinforcing fibers require a good compatibility of the glass fiber surface with the polymer matrix which is typically achieved via tailored surface coatings, also known as sizes. The application of a suitable size to the dried, recycled glass fiber agglomerates as obtained according to JP 2000034363A proved impossible. The fiber agglomerates treated with aqueous size were not able to be re-separated after drying and brought into a feedable form. In no case was the quality of virgin glass fiber able to be achieved using the process described in JP 2000034363A.

JP2000037726A also describes a process for removing glass fibers, here chopped glass fibers, when recycling polyamide 6 (PA6)-based composite plastics. JP2000037726A comprises initially depolymerizing the PA6 polymer matrix and thus separating it from the glass fibers. JP2000037726A additionally describes the in-principle possibility of also recovering the glass fibers by at the end of the depolymerization converting residual constituents of the PA6 employed as the matrix polymer remaining on the fibers into gaseous constituents by pyrolytic decomposition by heating to 400° C-700° C. The temperature necessary for pyrolysis must be supplied to the process from an external source. JP 2000037726A finally describes the option of subsequently heating the fibers “cleaned” in this way to temperatures above their melting temperature. This energy for melting the glass fibers must also be supplied from an external source.

Neither JP 2000034363A nor JP2000037726A are concerned with the fundamental problem of the additives additionally employed in the plastic, with the degradation products thereof or with the removal thereof from the washing water or the pyrolysis products. Yet the latter is necessary to prevent these often not environmentally unconcerning substances from passing into the environment.

Problem Addressed by the Present Invention

Starting from the prior art described hereinabove the problem addressed by the present invention is that of providing a process for recycling GRP, preferably polyamide 6-, polybutylene terephthalate (PBT)- and polyethylene terephthalate (PET)-based GRP, without the use of (washing) water for cleaning the glass fibers by means of which not only the (matrix) polymer in the form of its monomers but also the glass fibers in the form of glass suitable for glass fiber production may be recovered and in the polymerization process/the glass fiber production process be processed into virgin-quality polymer/glass fiber. At the same time additives shall be dischargeable from the recycling circuit effectively and without adverse effects on the environment and the process be sufficiently economic for large industrial scale application also to be economically viable.

It has surprisingly been found that in contrast to the teaching of the abovementioned prior art it is possible to produce from postconsumer GRP materials, in particular from glass fiber-reinforced thermoplastics based on PA6, PET or PBT, virgin quality glass fibers while also by depolymerization removing the majority of the polymer matrix which can be reconstructed to afford the identical original polymer.

Subject Matter of the Invention

The solution to the problem and thus the subject matter of the present invention is a process for recycling GRP by
a) adepolymerizing up to 80% by weight of the polymer matrix of a GRP, removing the cleavage products resulting from the polymer matrix and enriching the remaining residues in a mixture of glass-based component, residual matrix, monomer, cleavage products and constituents problematic for recycling and
b) utilizing the organic proportion remaining in the residue at the end of process step a) as an energy source by using its heat of combustion for heating and melting the glass-based component and simultaneously for removing the organic constituents by conversion into gaseous combustion products.

The polymer matrix/the cleavage products thereof are thus not quantitatively removed from the glass fibers in process step a).

According to the invention “problematic constituents” are in particular impurities and additives employed for the original intended use of the GRP.

Problematic constituents for the recycling of GRP's according to the invention are preferably functional additives, in particular UV stabilizers, heat stabilizers, gamma ray stabilizers, antistats, elastomer modifiers, flow promoters, demolding agents, flame retardants, emulsifiers, nucleating agents, plasticizers, lubricants, dyes, pigments or additives for increasing electrical conductivity. These and further additives are described, for example, in Gächter, Muller, Kunststoff-Additive [Plastics Additives], 3rd edition, Hanser-Verlag, Munich, Vienna, 1989 and in the Plastics Additives Handbook, 5th Edition, Hanser-Verlag, Munich, 2001.

Impurities in the context of the present invention are preferably degradation products of the additives and impurities, in particular dust, earths, iron oxides in the form of rust or foreign substances that have penetrated into the polymer matrix or adhere thereto.

The process according to the invention thus consists of two separate processes in which the components originally employed for producing GRP are separated into monomers and cleavage products on the one hand and into a glass melt on the other hand.

The process according to the invention also makes it possible to remove the constituents problematic for recycling, in particular impurities, and thus produce recyclates having virgin material character.

It is a feature of the process according to the invention that even passage through repeated usage-recycling cycles does not lead to substantial impairment of the product quality of the (matrix) polymer upon which the GRP is based when said polymer is re-synthesized from the monomers generated as cleavage products. In the process according to the invention the polymer matrix/the to-be-recycled (matrix) polymer is recycled to an extent of more than 50% by weight to about 80% by weight.

For clarity it should be noted that the scope of the present invention encompasses all reported definitions and parameters in general or in preferred ranges in any desired combinations. This applies not only to substance parameters but also to any forms of the use and the process such as are described in the context of the present invention. Unless otherwise stated the recited standards are to be understood as meaning the version valid on the filing date. Unless otherwise stated reported percentages are percentages by weight. In the context of the present invention the terms (matrix) polymer and polymer matrix have the same definition, wherein the focus/emphasis of the term polymer matrix is on the matrix while in the term (matrix) polymer the emphasis is on the polymer.

According to Kunststoffe.de, “Begriffsdefinitionen für das werkstoffliche Recycling”, excerpt from W. Hellerich, G. Harsch, E. Baur, Werkstoff-Führer Kunststoffe 10/2010, p. 55 at:

https://www.kunststoffe.de/themen/basics/recycling/werkstoffliches-recycling/artikel/begriffsdefinitionen-fuer-das-werkstoffliche-recycling-1001597.html

recyclate is an umbrella term which refers to a molding material/a processed plastic having defined properties. In many cases the recyclate is admixed with virgin material. A recyclate has in its history already undergone a manufacturing process. A masterbatch or a blend produced from two or more plastics by processing, i.e. by a manufacturing process, is not considered a recyclate.

Preferred Embodiments of the Invention

The present invention preferably relates to a process in which the GRP to be employed in process step a) is heated and optionally after addition of catalysts or depolymerization-promoting auxiliaries the polymer matrix of the GRP is subjected to cleavage in the absence of air.

The present invention therefore relates to a process in which during or after process step a) the cleavage products and/or the added auxiliaries, in particular hydrolysis or solvolysis liquids, are distilled off by pressure reduction.

The present invention preferably relates to a process for recycling GRP in which in process step a) at least 50% by weight of the polymer matrix is depolymerized.

The present invention particularly preferably relates to a process for recycling GRP in which in process step a) at least 50% by weight and at most 80% by weight of the polymer matrix is depolymerized.

In the case where process step b) is not performed in a furnace for glass production process step b) is followed in a further process step c) by removal of the glass melt for further processing.

Process Step a)

The depolymerization in process step a) is preferably performed with addition of auxiliaries or catalysts to promote the depolymerization. Preferred catalysts which promote the depolymerization of (matrix) polymers are bases or acids or salts thereof. Inorganic bases or inorganic acids or salts thereof are particularly preferred. It is very particularly preferable to employ calcium hydroxide, calcium carbonate, sodium carbonate, potassium carbonate or phosphoric acid. These catalysts which promote the depolymerization of (matrix) polymers are employed in concentrations in the range from 0.1% to 20% by weight, preferably in concentrations in the range from 0.5% to 10% by weight, particularly preferably in the range from 1% to 7% by weight, in each case based on the polymer matrix altogether introduced in process step a).

The polymer matrix of a GRP to be employed in the process according to the invention preferably contains essentially at least one polymer from the group of polyamide 6 (PA6), polyamide 66 (PA66), polyethylene terephthalate (PET), polybutylene terephthalate (PBT) or a copolymer of PET and PBT. Essentially is preferably to be understood as meaning to an extent of at least 70% by weight based on the polymer matrix introduced in process step a).

The polymer matrix of a GRP to be employed in the process according to the invention preferably contains essentially polyamide 6 (PA6), polyamide 66 (PA66), polybutylene terephthalate (PBT) or a copolymer of PBT and PET.

The polymer matrix of a GRP to be employed in the process according to the invention preferably contains essentially polyamide 6 (PA6) or polyamide 66 (PA66).

The polymer matrix of a GRP to be employed in the process according to the invention preferably contains essentially polybutylene terephthalate (PBT) or a copolymer of PBT and PET.

In the case of PA6 c-caprolactam is obtained as the depolymerization product in process step a). Depolymerization in process step a) preferably makes it possible to recover from PA6 50% to 80% by weight of the c-caprolactam originally employed for production thereof.

In the context of the research carried out for the present invention it was found that at commencement of a depolymerization carried out in process step a) of GRP based on glass fiber-reinforced PA6 high decomposition rates are achieved and few foreign substances are observed in the cleavage products. According to the invention the preferred cleavage product in the depolymerization of PA6 carried out in process step a) is c-caprolactam. Potassium carbonate or sodium carbonate in particular result in high yields of c-caprolactam.

Before use in the depolymerization the GRP components to be employed in process step a) are preferably collected in type-similar fashion and also employed in process step a) in type-similar fashion. Type-similar is to be understood as meaning that the plastics to be processed while identical in terms of their base polymers differ from one another in particular properties, for example flame retardant additives. See: Kunststoffe.de, “Begriffsdefinitionen für das werkstoffliche Recycling”, excerpt from W. Hellerich, G. Harsch, E. Baur, Werkstoff-Führer Kunststoffe 10/2010, p. 55 at:

https://www.kunststoffe.de/themen/basics/recycling/werkstoffliches-recycling/artikel/gegriffsdefinitionen-fuer-das-werkstoffliche-recycling-10015973.html

Before use in the depolymerization the GRP components to be employed in process step a) are particularly preferably collected in type-identical fashion and also employed in process step a) in type-identical fashion in order that the cleavage products removed in process step a) need not be subject to any costly and inconvenient workup.

In the context of the present invention type-identical is to be understood as meaning that plastics of identical designation according to DIN EN ISO 11469/VDA 260, optionally from different raw material producers, are processed. See: Kunststoffe.de, “Begriffsdefinitionen fur das werkstoffliche Recycling”, excerpt from W. Hellerich, G. Harsch, E. Baur, Werkstoff-Führer Kunststoffe 10/2010, p. 55 at:

https://www.kunststoffe.de/themen/basics/recycling/werkstoffliches-recycling/artikel/begriffsdefinitionen-fuer-das-werkstoffliche-recycling-1001597.html

The GRP components to be employed in process step a) are before use in the depolymerization preferably subjected to a cleaning to prevent adhering impurities from being introduced into the pyrolysis of the process step a) in the first place.

The GRP components to be employed in process step a) are before use in the depolymerization preferably shredded into small pieces to simplify/to accelerate the handling, conveyability and depolymerization of the (matrix) polymer. In the context of the present invention shredding is representative of any comminution processes, in particular mechanical comminution processes. Comminution processes arranged upstream of the process step a) according to the invention were scientifically investigated, for example in the project Recycling von Polymeren aus Schredderfraktionen, project partner UNISENSOR Sensorsysteme GmbH, Karlsruhe. DE 10 2014 111871 B1, which came from the project, relates to an apparatus and a corresponding process for separating one or more material fractions from at least one material stream of free-flowing bulk material, preferably from chunks of recyclable plastics. The content of DE 10 2014 111871 B1 is fully incorporated into the present application.

In a further preferred variant the shredded fraction is before the depolymerization in process step a) milled to afford particles having particle sizes<10 mm, particularly preferably<5 mm. The term milled material, used in this context, which is obtained by milling of plastic, especially preferably has different and irregular particle sizes in the range from 2 to 5 mm and may contain dust fractions. In this regard see: Kunststoffe.de, “Begriffsdefinitionen für das werkstoffliche Recycling”, excerpt from W. Hellerich, G. Harsch, E. Baur, Werkstoff-Führer Kunststoffe 10/2010, p. 55 at:

https://www.kunststoffe.de/themen/basics/recycling/werkstoffliches-recycling/artikel/begriffsdefinitionen-fuer-das-werkstoffliche-recycling-1001597.html

In a further preferred variant the shredded fraction is before the depolymerization in process step a) milled to afford powder having particle sizes<1 mm and the powder mixed with at least one depolymerization-promoting auxiliary and/or catalyst.

In one embodiment if required the fraction obtained from the shredding (shredded fraction) may be subjected to further workup before use in the depolymerization in process step a). Metal components adhering to the (matrix) polymer may preferably be removed using additional process steps and recycled separately. In this case it is preferable to use magnetic separators or induction separators.

The choice of the at least one depolymerization-promoting catalyst and/or auxiliary is preferably effected such that these undergo residueless combustion in process step b) to afford energy or can remain as inorganic constituents in the glass component and ultimately in the recovered glass without having a noticeable effect on glass quality.

It is preferable when in process step a) at least 20% by weight of the original polymer matrix remains as organic residue. This remaining at least 20% by weight of polymer matrix is utilized in process step b) in order via the combustion process and the resulting heat of combustion to melt the glass-based component, preferably glass fibers.

It is preferable when this simultaneously frees the glass-based component of organic impurities. It is preferable when GRP components based on easily and rapidly depolymerizable polymers are employed in process step a). Preferred easily and rapidly depolymerizable polymers in the context of the present invention are polybutylene terephthalate (PBT), polyethylene terephthalate (PET) or polyamide 6 (PA6).

In the case of PA6-based GRP proportions the depolymerization is preferably performed by heating in the absence of oxygen, particularly preferably in the presence of at least one basic catalyst at temperatures<350° C.; in this case the depolymerization may be referred to as a pyrolysis.

In the case of PBT-based GRPs to be recycled the depolymerization in process step a) is preferably performed in the presence of water as the depolymerization-promoting auxiliary. The depolymerization of PBT-based GRP is preferably performed at temperatures in the range from 240° C. to 350° C. This forms terephthalic acid and 1,4-butanediol/the dehydration product thereof tetrahydrofuran. It is likewise possible to perform the depolymerization in the presence of alcohols as auxiliaries in the form of a solvolysis which results in the formation of the corresponding esters.

In the case of PET-based GRPs to be recycled the depolymerization is preferably performed in the presence of water and/or alcohols at temperatures above 280° C.

Preferably and in one embodiment the cleavage products of the GRP (matrix) polymer generated in the process step a) by cleavage of the polymer chains are supplied, preferably after any necessary purification, especially by distillation, to a subsequent repolymerization to produce a recyclate plastic having virgin material character. Re-polymerization of the cleavage products makes it possible, especially after additional purification of these cleavage products referred to as monomers, to reproduce the same polymer/the same plastic, in particular to produce new GRP based on recyclate. This process variant is preferably employed in the cases in which the GRP (matrix) polymer contains only few monomers and ideally only one monomer and the obtained monomer(s) is or are separable and purifiable by processes established on a large industrial scale, preferably by distillations or rectifications.

GRPs especially preferable for workup by the process according to the invention are those based on PA6 as the (matrix) polymer which is in turn based on c-caprolactam as the monomer.

Optionally or in a preferred embodiment before re-polymerization to produce a recyclate plastic the monomers/cleavage products generated in process step a) are sent to large industrial scale processing plants and purified together with monomers classically produced by petrochemical means. Here too, preferred large industrial scale processing plants are distillation plants or rectification plants.

Preferably and in a further embodiment proportions of the monomers/cleavage products of the GRP (matrix) polymer generated in process step a) by cleavage of the polymer chains are also employed as additional fuel for the combustion operation in process step b). This process variant is preferably employed in cases in which the GRP (matrix) polymer is constructed from at least two different monomers or the polymer matrix consists of a blend of at least two different plastics or else a type-identical postconsumer plastic as a feed stream is not available. Feed stream is an established term in process engineering. This refers to an inflow (feed) of reactants into a process, in the present case the process according to the invention for recycling GRP.

The depolymerization, which depending on the type of the underlying (matrix) polymer is preferably to be understood as meaning a hydrolysis, solvolysis or pyrolysis/thermolysis, may be performed in different process engineering apparatuses.

GRPs to be employed according to the invention, in particular polyamide-based GRPs, are preferably directly heated in the absence of oxygen, preferably under a nitrogen atmosphere, (pyrolysis/thermolysis) after addition of suitable auxiliaries/catalysts, in particular basic catalysts. This is preferably done using batch reactors which through temporally staggered startup can provide material for the second process step b) in a quasi-continuous fashion.

In the cases in which the (matrix) polymer is depolymerized in process step a) via superheated steam or using alcohols, in particular PBT or PET, it is preferable to employ high-pressure autoclaves. After an exposure time the volatile components are distilled off, preferably under reduced pressure, and the remaining residue transferred to process step b).

Process Step b)

Process step b) is preferably carried out in a rotary kiln—see U. Richers, Thermische Behandlung von Abfällen in Drehrohröfen, Forschungszentrum Karlsruhe GmbH, Karlsruhe, 1995.

It is preferable when in process step a) not more than 20% by weight of the original (matrix) polymer remains as organic residue which, together with the glass-based component, is supplied to the process step b). This organic residue is combusted in process step b), wherein the heat of combustion initially heats and ultimately melts the glass-based component. Process step b) is preferably performed at temperatures in the range from 1300° C. +/−300° C.

By combustion of the remaining organic material the impurities, additives and decomposition products remaining on the glass-based component are simultaneously removed. Oxidation of organic impurities, additives and decomposition products to CO₂ and water is preferably effected.

It is preferable when the entirety of the energy for the process step b) is generated from the combustion of the organic material/residue introduced into process step b) with the glass-based component.

However, in one embodiment additional energy is supplied in process step b), preferably using conventional gas burners. In a preferred embodiment these are supplied with gaseous fuels based on C₁-C₄-hydrocarbons as combustion fuel. It is preferable to employ natural gas or biogas for this purpose.

The supplying of additional energy in process step b) should preferably be used when the heat of combustion of the organic material/residue or of the polymer still present in the organic residue is insufficient to achieve the melting temperature of the glass-based component and/or to bridge the customary residence time of the glass-based component in the molten state until further processing.

The combustion of the organic material/residue in process step b) is preferably carried out by supplying air, air-oxygen mixtures or pure oxygen.

The process step b) is preferably performed directly in the melting region of a glass fiber production plant, wherein the organic material/the organic residue are combusted using additionally introduced air/oxygen. The residue obtained in process step a) is fed into the melting region of the furnace of the glass fiber production plant as a sidestream to the main feed stream of the inorganic glass raw material mixture or optionally supplied to the furnace of the glass fiber production plant as a solid after comminution, in particular pulverization. The solids feeding may be carried out separately or in admixture with the main feed stream of the glass raw material mixture.

It is likewise preferable when process step b) is performed in immediate spatial proximity to a glass fiber production plant and the glass melt resulting from process step b) is directly combined with the glass melt of the glass fiber production plant.

In the case where the glass-based component of the GRP is glass fibers the combustion operation in process step b) also causes impurities on the surface of the glass fiber or sizes to be oxidized and discharged via the combustion gases, preferably in the form of CO₂.

Impurities or decomposition products from the polymer matrix of the employed GRP which cannot be oxidized to CO₂ in process step b) are preferably discharged via the combustion gases.

Impurities, additives or decomposition products thereof may form harmful substances in the process step b). These are preferably supplied together with the generated combustion gases to an offgas purification where the harmful substances are intercepted to meet legislative imissions regulations.

Especially in the case of a combustion of a (matrix) polymer containing bromine- or phosphorus-containing additive residues carried out in process step b) it is possible in principle for toxic byproducts undesirable for man and the environment to be formed and thus be present in the combustion gases. However, as a result of modern offgas purification it is nowadays readily possible to separate these from the offgas so that these substances do not pass into the environment and legislative emissions provisions are met. The German Federal Immissions Control Act (law relating to protection from harmful environmental effects from air pollution, noise, vibration and similar occurrences), especially in its latest version of Apr. 12, 2019, (BGBI I p. 432) regulates an important branch of environmental law and is the practice-relevant regulatory framework for the protection of man, animals, plants, soil, water, atmosphere and cultural assets from immissions and emissions.

In one process variant of the process step b) organic (matrix) polymer adhering to the glass-based components, preferably glass fibers, is initially pyrolyzed at elevated temperature and then the heat of combustion of the carbonization residues adhering to the glass constituents/glass fibers and the heat of combustion of the generated pyrolysis gas and/or pyrolysis oil are together utilized for melting the glass constituents/glass fibers.

It is particularly preferable to introduce pyrolysis gases generated in process step b) at another point in the melting operation in the process according to the invention. It is likewise preferable to mix the pyrolysis gases directly with the natural gas preferred for use for the melting operation in process step b). This process variant makes it possible, even after a complete depolymerization of the (matrix) polymer in the process step a), to still provide sufficient heat of combustion in process step b) to melt the glass-based component, preferably glass fibers, and maintain it at melting temperature until further processing.

In the case where process step b) is carried out directly and immediately in the melting region of a glass fiber production plant it is preferable to introduce conventional glass fiber additives, in particular SiO₂, Al₂O₃, MgO, B₂O₃, CaO, into the composition of glass/matrix residues generated from process step a).

Process Step c)

If process step b) is not already performed in or in the spatial vicinity of a furnace for glass production the removal of the glass melt for subsequent further processing is carried out in a process step c).

It is preferable when the glass melt generated in process step c) is supplied to a production of glass fibers or a production of glass powders or glass spheres. It is particularly preferable when the glass component generated in process step c) is supplied to a conventionally operated furnace for production of glass fibers in order then to be available for re-spinning into glass fibers.

Since virtually all impurities are concentrated in the organic residue at the end of process step a) and via the subsequent combustion process in process step b) discharged in the form of combustion gases and thus removed from the glass constituents the process according to the invention makes it possible to produce a high-quality glass melt from which in turn high-quality glass recyclates, in particular glass recyclates in the form of glass fibers, milled glass or glass powder, may be produced in further processing steps.

In the preferred case of depolymerization/pyrolysis of the polymer matrix in process step a) to an extent of not more than 80% by weight of the original GRP matrix, sufficient organic material remains on the glass constituents for the combustion thereof to provide a sufficient heat of combustion to melt the glass-based component of the GRP, preferably glass fibers, and supply it to a mechanical recycling. Using the not more than 20% by weight of organic material remaining from the original (matrix) polymer, impurities, additives and decomposition products remaining on the glass-based component are oxidized to CO₂ in the combustion process and thus removed.

Especially Preferred Embodiments of the Invention

The present invention especially relates to a process for recycling GRP by

a) depolymerizing up to 80% by weight of the polymer matrix of a GRP, removing the cleavage products resulting from the polymer matrix and enriching the remaining residues in a mixture of glass-based component, residual matrix, monomer, cleavage products and constituents problematic for recycling and

b) utilizing the organic proportion remaining in the residue at the end of process step a) as an energy source by using its heat of combustion for heating and melting the glass-based component and simultaneously for removing the organic constituents by conversion into gaseous combustion products with the proviso that the polymer matrix is based on polyamide 6 (PA6), on polyamide 66 (PA66), on polybutylene terephthalate (PBT) or on a copolymer of PBT and PET.

The present invention especially additionally relates to a process for recycling GRP by

a) depolymerizing up to 80% by weight of the polymer matrix of a GRP, removing the cleavage products resulting from the polymer matrix and enriching the remaining residues in a mixture of glass-based component, residual matrix, monomer, cleavage products and constituents problematic for recycling and

b) utilizing the organic proportion remaining in the residue at the end of process step a) as an energy source by using its heat of combustion for heating and melting the glass-based component and simultaneously for removing the organic constituents by conversion into gaseous combustion products with the proviso that the polymer matrix is based on polyamide 6 (PA6) or on polyamide 66 (PA66), in particular PA6.

The present invention especially also relates to a process for recycling GRP by

a) depolymerizing up to 80% by weight of the polymer matrix of a GRP, removing the cleavage products resulting from the polymer matrix and enriching the remaining residues in a mixture of glass-based component, residual matrix, monomer, cleavage products and constituents problematic for recycling and

b) utilizing the organic proportion remaining in the residue at the end of process step a) as an energy source by using its heat of combustion for heating and melting the glass-based component and simultaneously for removing the organic constituents by conversion into gaseous combustion products with the proviso that the polymer matrix is based on polybutylene terephthalate (PBT) or on a copolymer of PBT and PET.

The present invention especially also relates to a process for recycling GRP by

a) depolymerizing up to 80% by weight of the polymer matrix of a GRP, removing the cleavage products resulting from the polymer matrix and enriching the remaining residues in a mixture of glass-based component, residual matrix, monomer, cleavage products and constituents problematic for recycling and

b) utilizing the organic proportion remaining in the residue at the end of process step a) as an energy source by using its heat of combustion for heating and melting the glass-based component and simultaneously for removing the organic constituents by conversion into gaseous combustion products and

c) separating the glass melt for further processing with the proviso that the polymer matrix is based on polyamide 6 (PA6), on polyamide 66 (PA66), on polybutylene terephthalate (PBT) or on a copolymer of PBT and PET.

The present invention especially further relates to a process for recycling GRP by

a) depolymerizing up to 80% by weight of the polymer matrix of a GRP, removing the cleavage products resulting from the polymer matrix and enriching the remaining residues in a mixture of glass-based component, residual matrix, monomer, cleavage products and constituents problematic for recycling and

b) utilizing the organic proportion remaining in the residue at the end of process step a) as an energy source by using its heat of combustion for heating and melting the glass-based component and simultaneously for removing the organic constituents by conversion into gaseous combustion products and

c) separating the glass melt for further processing with the proviso that the polymer matrix is based on polyamide 6 (PA6) or on polyamide 66 (PA66), in particular PA6.

The present invention especially finally relates to a process for recycling GRP by

a) depolymerizing up to 80% by weight of the polymer matrix of a GRP, removing the cleavage products resulting from the polymer matrix and enriching the remaining residues in a mixture of glass-based component, residual matrix, monomer, cleavage products and constituents problematic for recycling and

b) utilizing the organic proportion remaining in the residue at the end of process step a) as an energy source by using its heat of combustion for heating and melting the glass-based component and simultaneously for removing the organic constituents by conversion into gaseous combustion products and

c) separating the glass melt for further processing with the proviso that the polymer matrix is based on polybutylene terephthalate (PBT) or on a copolymer of PBT and PET.

### EXAMPLES

150 g of shredded GRP made of Durethan® BKV30H2.0 from Lanxess Deutschland GmbH were in a glass apparatus with a KPG stirrer (blade stirrer & torque measurement) melted in a 500 mL round bottom flask in a metal bath (T=320° C.). 5% potassium carbonate, based on the postconsumer plastic, was added as a finely powdered depolymerization catalyst.

The melting process was performed statically and stirred only periodically (approx. every 5 minutes) with about 2 to 3 revolutions. The melting of the 150 g of shredded GRP was completed after approx. 40 minutes.

With slow stirring at 12 revolutions per minute (rpm) the internal pressure was reduced to 20 to 30 mbar in 50 mbar steps and considerable foam development was observed.

The polyamide 6 starting material caprolactam was converted into the gaseous state and the entire apparatus was continuously heated using a hot air blower in order that this monomer having a melting point of 68° C. did not pass into the solid state and cause blockages in the apparatus.

The following fractions of caprolactam shown in table 1 were obtained:

TABLE 1

| FLASK | Duration (min) | Amount (g) |
|---|---|---|
| 1 | 32 | 43.14 |
| 2 | 25 | 40.34 |
| 3 | 44 | 39.42 |
| Sum | 101 | 82.90 |
| Yield | | 79% |

The caprolactam fractions 1 to 3 were analyzed by gas chromatograph. The purity decreased from fraction 1 to fraction 3 but in each case proved suitable for repolymerization by hydrolytic polymerization. The caprolactam proportion in these fractions was over 99.5% by weight!

The residue remaining after depolymerization was portioned and transferred to a glass boat, therein surrounded by pure oxygen in a muffle furnace, ignited using a Bunsen burner and combusted without further heating.

At the end of the combustion process the glass residue was isolated, dried at 115° C. according to DIN 52331 and homogenized. The samples were then subjected to ICP-OES analysis.

Except for a measured CuO concentration which was due to the heat stabilization of the employed Durethan® grade there were no noticeable deviations in the glass composition of the glass residue compared to the glass fiber used in Durethan® BKV30H2.0.

1. A process for recycling glass fiber-reinforced plastics (GRP), comprising the steps of
a) depolymerizing up to 80% by weight of the polymer matrix of glass fiber-reinforced plastics, removing the cleavage products resulting from the polymer matrix and enriching the remaining residues in a mixture of glass-based component, residual matrix, monomer, cleavage products and constituents problematic for recycling and
b) utilizing the organic proportion remaining in the residue at the end of process step a) as an energy source by using its heat of combustion for heating and melting the glass-based component and simultaneously for removing the organic constituents by conversion into gaseous combustion products.

2. The process as claimed in claim 1, wherein at least 50% by weight of the polymer matrix, is depolymerized.

3. The process as claimed in claim 1, wherein the glass fiber-reinforced plastics be employed in process step a) are previously collected in type-similar, fashion and employed in process step a) in type-similar, fashion.

4. The process as claimed in of claim 1, wherein the glass fiber-reinforced plastics components to be employed in process step a) are shredded into small pieces before use in the depolymerization.

5. The process as claimed in of claim 1, whereinthe glass fiber-reinforced plastics to be employed in process step a) are subjected to a cleaning before the depolymerization.

6. The process as claimed in of claim 1, wherein the glass fiber-reinforced plastics to be employed in process step a) is heated and after addition of additives subjected to pyrolytic cleavage in the absence of air.

7. The process as claimed in of claim 1, wherein the glass fiber-reinforced plastics to be employed in process step a) is depolymerized in the presence of water or alcohols.

8. The process as claimed in of claim 1, wherein during or after process step a) the cleavage products and/or the added hydrolysis/solvolysis liquids are distilled off by pressure reduction.

9. The process as claimed in of claim 1, wherein in process step a) at least 20% by weight of the original polymer matrix remains in the organic residue.

10. The process as claimed in of claim 1, wherein the cleavage products of the glass fiber-reinforced plasticsGRP (matrix) polymer matrix generated in process step a) by cleavage of the polymer chains are supplied to a subsequent repolymerization to produce a recyclate plastic having virgin material character.

11. The process as claimed in of claim 1, wherein the cleavage product is ε-caprolactam and the recyclate plastic is polyamide 6.

12. The process as claimed in one or more of claim 1, wherein the cleavage products are terephthalic acid and 1,4-butanediol/the dehydration product thereof tetrahydrofuran and the recyclate plastic is polybutylene terephthalate.

13. The process as claimed in of claim 1, wherein the cleavage products of the glass fiber-reinforced plastics polymer matrix generated in process step a) by cleavage of the polymer chains are at least in part also employed as fuel for the combustion operation in process step b).

14. The process as claimed in of claim 1, wherein the remaining at least 20% by weight of organic material, impurities, additives and decomposition products remaining on the glass constituents are removed from the glass reinforcement/glass melt in the combustion process in process step b)

15. The process as claimed in of claim 1, wherein additional energy is supplied in process step b).

16. The process as claimed in claim 2, at least 50% by weight and at most 80% by weight of the polymer matrix is depolymerized.

17. The process as claimed in claim 3, wherein the glass fiber-reinforced plastics to be employed in process step a) are previously collected in type-identical fashion and employed in process step a) in type-identical fashion.

18. The process as claimed in claim 1, wherein the remaining at least 20% by weight of organic material, impurities, additives and decomposition products remaining on the glass constituents are converted to CO₂ by oxidation.

19. The process as claimed in claim 15, wherein additional energy is supplied in process step b) by means of a gas burner.
